(19) United States
(12) Patent Application Publication
(10) Pub. No.: US 2012/0022504 A1
(43) Pub. Date: Jan. 26, 2012

(54) DEVICE, APPARATUS, AND METHOD OF ADIPOSE TISSUE TREATMENT

(75) Inventors: Haim Epshtein, Benyamina (IL); Boris Vaynberg, Zichron Yaakov (IL); James Bartholomeusz, Beverly Hills, CA (US)

(73) Assignee: SYNERON MEDICAL LTD., Yoqneam Illit (IL)

(21) Appl. No.: 12/665,916

(22) PCT Filed: Jul. 12, 2009
(86) PCT No.: PCT/IL2009/000695

§ 371 Date: Aug. 10, 2011

Related U.S. Application Data
(60) Provisional application No. 61/095,973, filed on Sep. 11, 2008.
(60) Provisional application No. 61/107,744, filed on Oct. 23, 2008.

(51) Int. Cl. A61B 18/22; A61N 5/067; A61B 18/18; A61M 1/00

(52) U.S. Cl. 604/542; 606/15; 607/89; 606/33

(57) ABSTRACT

A method and apparatus for adipose tissue treatment whereby two types of electromagnetic radiation are applied to the volume of tissue to be treated, One type of the electromagnetic radiations being RF and the second type of electromagnetic radiation being visible or infrared radiation.

The present application is a national phase application being filed under 37 CFR 371 and based on Patent Cooperation Treaty filing PCT/IL2009/000695, which claims priority to United States Provisional Application for Patent filed on Aug. 1, 2008 and assigned Ser. No. 61/085,424 and, the present application is a continuation-in-part of the United States patent application that was assigned Ser. No. 12/357,564, filed on Jan. 22, 2009 and attributed to the same inventors and the same assignee, which application claims priority to the United States Provisional Application for patent that was filed on Jan. 24, 2008 and assigned Ser. No. 61/023,194.

### TECHNICAL FIELD

The present device, apparatus, and method relate to the field of adipose tissue treatment and aesthetic body sculpturing.

### BACKGROUND

Liposuction is a popular technique for removal of fat from different sites of a subject's body. The process changes the external contours of the body and sometimes is described as body sculpturing. The fat is removed by a suction device via a cannula inserted into the appropriate site of the body. The process is painful and sometimes causes excessive bleeding.

Recently, improvements have been realized in liposuction procedures by the utilization of electro-magnetic energy or radiation such as an infrared laser radiation delivered through a fiber inserted into a cannula introduced into the treatment site. Laser radiation liquefies the adipose tissue. The liquefied tissue is either removed by suction or left in the subject body, where it gradually dissipates in a uniform way. Laser assisted liposuction is considered to be a more advanced and less invasive procedure when compared to traditional liposuction techniques.

For proper treatment, laser assisted liposuction requires application of high power ten to fifty watt laser energy or radiation. The radiation is applied in a continuous or pulse mode for relatively long periods. Sometimes more than one laser is used on the same treated tissue volume to speed up the treatment. Each of the lasers may operate in a different mode. For example, one of the lasers heats the target tissue volume, and the other one introduces laser power sufficient to destroy the adipose tissue in the same volume. This increases the cost of the equipment and prolongs the treatment session time. In addition, frequent cleaning and maintenance of the fiber tip from process debris will be required. All of the above slows down the treatment process, and in addition affects comfort and cost of procedure to the treated subject.

The industry would welcome a better solution to these and other existing problems.

### BRIEF SUMMARY

A method and apparatus for adipose tissue treatment where two types of electromagnetic radiation or energy are applied to the volume of tissue to be treated. One type of the electromagnetic energy is RF and the second type of electromagnetic energy is provided by visible or infrared radiation.

In some embodiments, both types of electromagnetic energy are delivered to the target volume subcutaneously by a light guide or needle that includes electrodes. In other embodiments, only one type of energy may be delivered to a target volume.

In some embodiments, the RF energy is delivered to a target volume of the tissue by an electrode applied to the skin. The energy delivered by the visible or infrared radiation is delivered subcutaneously by a needle, which is introduced into the same target volume of the tissue.

### BRIEF LIST OF DRAWINGS

The disclosure is provided by way of non-limiting examples only, with reference to the accompanying drawings, wherein:

FIG. 1 is a schematic illustration of the first exemplary embodiment of an electromagnetic energy-conveying needle.

FIGS. 2A-2C, collectively referred to as FIG. 2, are schematic illustrations of a number of cross sections of some of the exemplary embodiments of the needle of FIG. 1.

FIGS. 3A and 3B, collectively referred to as FIG. 3, are schematic illustrations of a second exemplary embodiment of an electromagnetic energy-conveying needle.

FIGS. 4A-4C, collectively referred to as FIG. 4, are schematic illustrations of a third exemplary embodiment of an electromagnetic laser energy-conveying needle.

FIGS. 5A-5C, collectively referred to as FIG. 5, are schematic illustrations of a fourth exemplary embodiment of an electromagnetic energy-conveying needle.

FIGS. 6A-6C are schematic illustrations of a fifth exemplary embodiment of an electromagnetic energy-conveying needle.

FIGS. 7A-7C, collectively referred to as FIG. 7, are schematic illustrations of a sixth exemplary embodiment of an electromagnetic energy-conveying needle.

FIG. 8 is a schematic illustration of an exemplary embodiment of an apparatus for laser and RF assisted liposuction employing the present needle.

FIGS. 9A-9D are schematic illustrations of additional exemplary embodiments of an electromagnetic energy-conveying needle.

FIG. 10 is a schematic illustration of the seventh exemplary embodiment of a laser radiation-conveying needle

FIGS. 11A and 11B are schematic illustrations of another exemplary embodiment of an apparatus for laser and RF assisted liposuction employing the present needle.

### DETAILED DESCRIPTION

The principles and execution of the needle, apparatus, and method described thereby may be best understood by reference to the drawings, wherein like reference numerals denote like elements through the several views and the accompanying description of non-limiting, exemplary embodiments.

The term “needle,” as used in the text of the present disclosure means a flexible or rigid light guide configured to be inserted during use into the subject tissue in order to deliver laser energy to a target volume of adipose tissue. In certain embodiments, the needle can be equipped with electrodes and configured during operation to apply RF energy to the treated tissue. The needle can also be configured to conduct a fluid to any part of the needle, and liquefied fat and the fluid from the target volume may be withdrawn. The needle may be a disposable or reusable needle.

The term “tissue” or “skin” as used in the text of the present disclosure means the upper tissue layers, such as epidermis, dermis, adipose tissue, muscles, and deeper located fat tissue.

The term “adipose tissue” used herein may also encompass, fat, and other undesirable tissue elements. The term “adipose tissue” is an example of undesirable or excessive tissue, but it should also be understood that the processes and treatments disclosed are applicable to other classes of tissue.

The term “tissue treatment,” as used in the present disclosure means application of one or more types of energy to the tissue to alter the tissue or obtain another desired treatment effect. The desired effect may include at least one of adipose tissue destruction, shrinking, breakdown, and skin tightening, haemostasis, inducing fat cells necrosis, inducing fat cells apoptosis, fat redistribution, adiposities (fat cell) size reduction, and cellulite treatment.

The terms “light,” “laser energy,” and “laser radiation” in the context of the present disclosure have the same meaning.

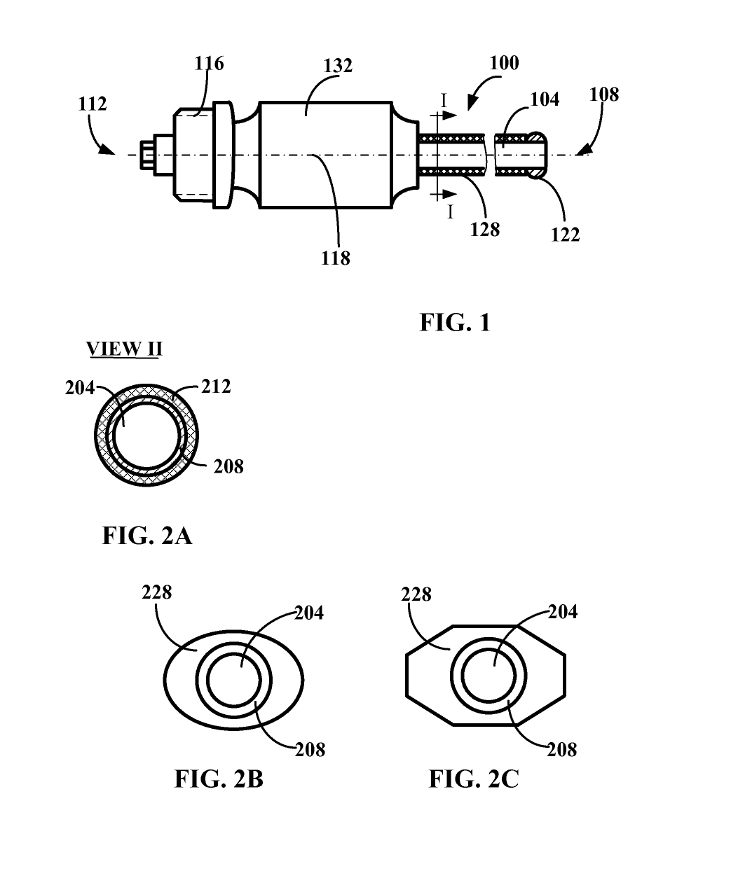

Reference is made to FIG. 1, which is a schematic illustration of a first exemplary embodiment of an electromagnetic radiation-conveying needle. Needle 100 is a needle shaped solid or hollow light conducting guide 104 having a first 108 end and a second end 112. First end 108 can be shaped for piercing the skin of a subject (not shown). The second end 112 is adapted to connect directly to a source of laser radiation by means of a connector (not shown) similar to a fiber optics type connector, for example SMA type connector and additional cable. Adjacent to first end 108 of needle 100 a mono-polar RF (Radio Frequency) electrode 122 is located and connected through the same connector 116 to a source of RF energy (not shown), which is a type of electromagnetic energy. Electrode 122 may connect to the source of RF energy, operating in frequency range of 100 KHz to 100 MHz, by a conventional conductive wire or specially deposited leads terminating at connector 116 over which for isolation purposes a protective coating or jacket 128 may be placed. Electrode 122 may be a thin metal sleeve or a ring having rounded angles stretched over first end 108 of needle 100 and fixed by any known means. The length of electrode 122 may be 1 to 50 millimeter depending on the type of treatment applied. Alternatively, electrode 122 may be electrochemically deposited on first end 108 of needle 100. Electrode 122 may be located adjacent to the first end of needle 100 such that first end 108 of needle 100 would protrude from electrode 122 or reside inside electrode 122.

First end 108 of needle 100 may be shaped for piercing the skin of a subject and may be terminated by a plane perpendicular to the optical axis 118 or at an angle to the optical axis 118 of needle 100. Alternatively, end 108 may have a radius or an obtuse angle. Other shapes of needle end 108 that improve either subject skin penetration properties, facilitate needle movement inside fibrotic fatty tissue, or laser power delivery quality are possible. In some cases, the skin incision is made by any well-known surgical means and the needle is introduced into the tissue. In an alternative embodiment laser radiation emitted through the first end 108 of needle 100, assists needle 100 into skin penetration process by providing continuous or pulsed laser power suitable for skin incision. Numeral 132 designates a handle by which the caregiver or person providing treatment holds and operates the needle. Handle 132 may include certain knobs for initiating or terminating treatment related processes. The length of needle 100 may vary from a few millimeters to a few hundred millimeters.

FIGS. 2A-2C, collectively referred to as FIG. 2, are schematic illustrations of a number of cross sections of some of the exemplary embodiments of the needle of FIG. 1. FIG. 2A is an exemplary cross section of needle 100 that has a round cross section. Needle 100 includes a solid light conducting core 204, a cladding 208 having a refractive index lower than core 204, and a protective jacket 212 that mechanically protects the sensitive surface of the needle. The diameter of core 204 may be 100 micron to 1500 micron, the diameter of cladding 208 may be 110 micron to 2000 micron, and the size of jacket 212 may be 200 micron to 2500 micron. Connection of needle body 104 to connector 116 may be performed by crimping or any other means known and established in the fiber optics industry.

In some embodiments, shown in FIGS. 2B and 2C, jacket 228 may have an elliptical or polygonal shape. These shapes provide different stiffness along the short and long symmetry axes of the needle cross section, and facilitate introduction and movement of the needle into the subject body.

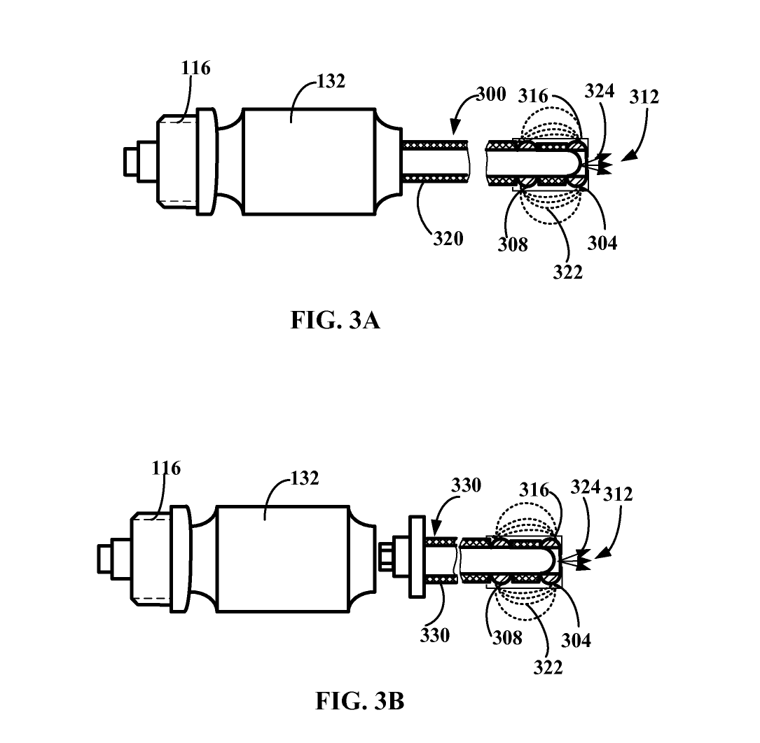

FIGS. 3A and 3B, collectively referred to as FIG. 3, are schematic illustrations of a second exemplary embodiment of an electromagnetic energy-conveying needle. These figures illustrate a needle 300 with bipolar electrodes 304 and 308 located adjacent radiation or energy emitting end 312 of needle 300. Electrodes 304 and 308 may be in a conductive coupling with the tissue of the treated subject or may be coated by a dielectric layer 316 and be in a capacitive coupling with the treated subject tissue. Electrodes 304 and 308 may be produced in a way similar to the one described above. FIG. 3 shows an exemplary embodiment of needle 300 with laser radiation emitting end 312 implemented as a spherical end. Other laser radiation emitting end 312 terminations are possible. Numeral 320 marks the fiber optics guide jacket. FIG. 3A illustrates a disposable or reusable needle 300 that includes handle 132. FIG. 3B illustrates a disposable or reusable needle 330 that in use is attached to handle 132. Numeral 322 marks RF current and numeral 324 marks the emitted laser radiation.

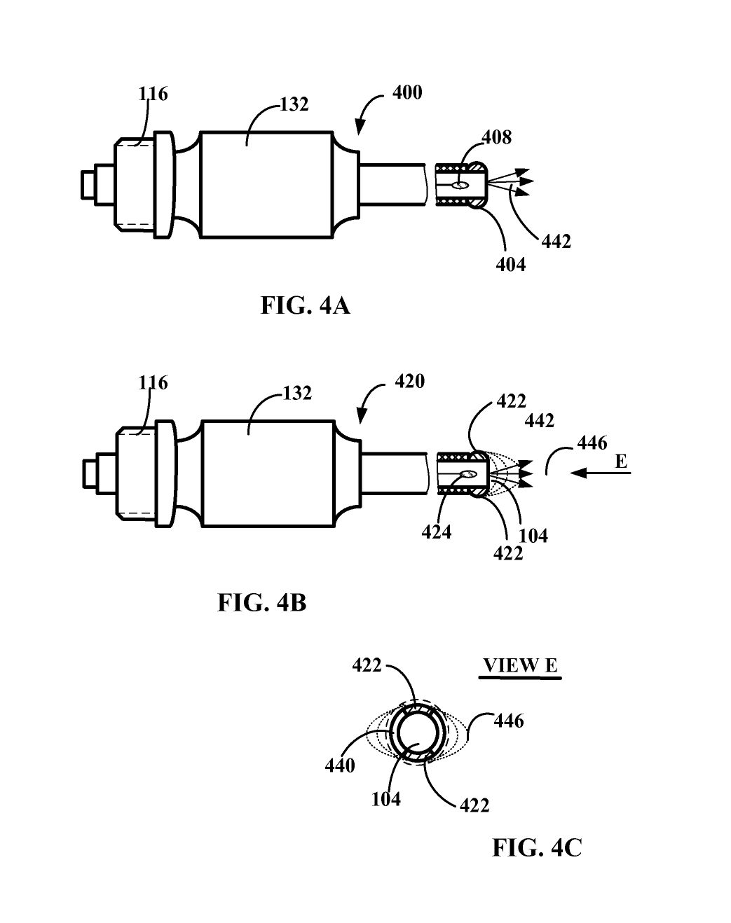

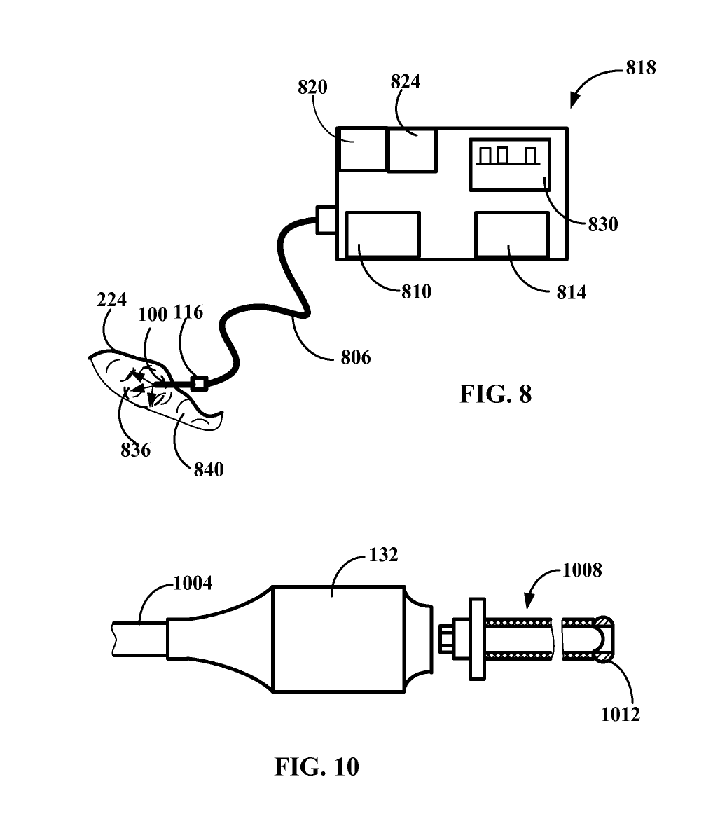

FIGS. 4A-4C, collectively referred to as FIG. 4, are schematic illustrations of a third exemplary embodiment of an electromagnetic radiation-conveying needle. Needle 400 (FIG. 4A) includes a mono-polar electrode 404 and a temperature sensor 408 that measures temperature in the target tissue volume. Knowledge of the temperature in the target tissue volume helps in informing caregiver on the treatment status and in establishing proper feedback to controller 818 (FIG. 8) and setting appropriate treatment parameters.

FIG. 4B is an illustration of a needle 420 with two electrodes 422 and temperature sensor 424. Electrode 404 (mono-polar) or electrodes 422 (bi-polar) may be implemented as one or more conductive rings or as a film deposited on one or both (opposite) sides of needle 420 circumference. Lines 446 indicate the current induced by bi-polar electrodes in the tissue and numeral 442 marks emitted by the needle laser radiation.

FIG. 4C is a view illustrating the radiation-emitting end of needle 420 with bi-polar electrodes 422 at least partially conforming to the needle shape. The electrodes may be made of foil, wire, thin metal plates, or electrochemically deposited. A temperature sensor 424 may also be placed on guide 104. An optional layer of a dielectric or isolator to avoid crosstalk or potential short circuit between the electrodes may coat the electrodes. Numeral 440 marks isolation between electrodes 422, which may be part of the dielectric coating or similar material. Changing the size of electrodes, (the size of the segment conforming to the needle shape) allows the volume of affected RF tissue to be changed.

In a bi-polar RF electrode configuration, an additional treatment progress status feedback method may be implemented. When RF energy is supplied to electrodes 422 it induces a current flow shown schematically by phantom lines 446 in the tissue between electrodes. It is known that tissue conductivity is temperature dependent. Accordingly, measuring the RF induced current value provides information on treated tissue status and allows the power and time of each of the laser radiation 442 or RF energy supplied to the target skin/tissue volume to be regulated.

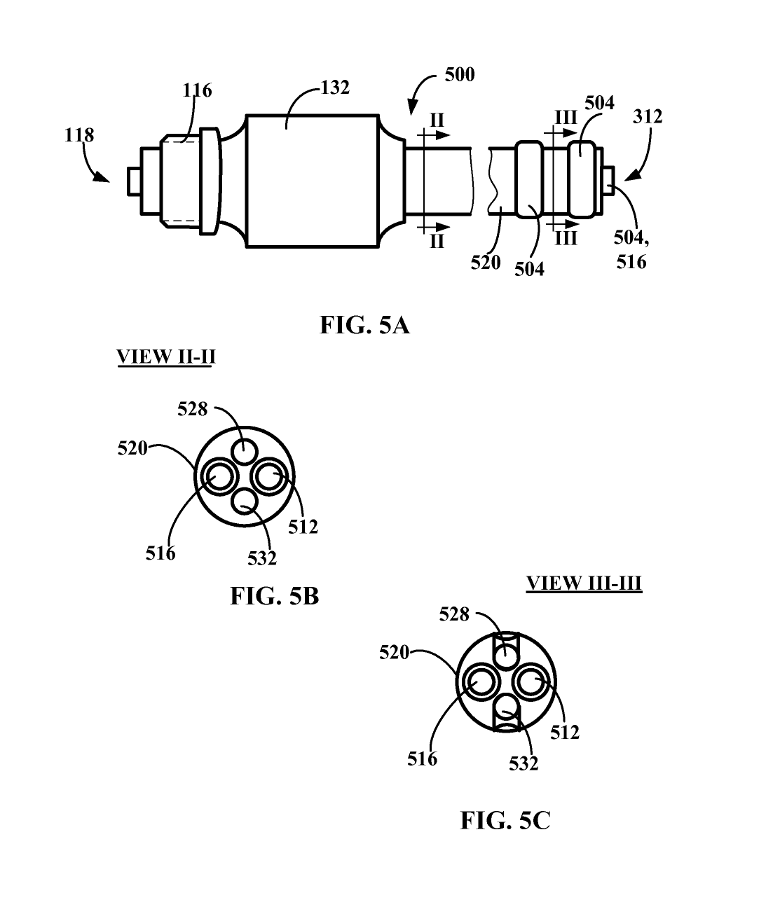

FIGS. 5A-5C, collectively referred to as FIG. 5, are schematic illustrations of a fourth exemplary embodiment of an energy-conveying needle 500 with RF energy supplying electrodes 504 and two light conducting guides 512 and 516. Both the RF energy-supplying electrodes 504 and light conducting guides 512 and 516 are incorporated into a connecting member 520 forming a single catheter like structure. RF electrodes 504, which may be rings of biocompatible conductive material, are tightened or deposited over the connecting member 520, which may be made from isolating material. One or more fluid conducting channels 528 and 532 may be made in connecting member 520. For example, fluids delivered through fluid delivery channel 528 may be used for cooling or heating the electrodes, or any other desired part of the needle or tissue, conductive fluids may be introduced into the treated tissue volume through channel 528, and other fluids. Adipose tissue treatment products and the fluid supplied to the tissue may be removed through fluid removal channel 532. In some embodiments, their may be one fluid conducting channel only and it may be used either for different fluids delivery to the treated volume or adipose tissue treatment products removal. There may be a switching arrangement switching as required the same channel between the two processes.

Channel 532 connects to a facility for adipose tissue laser treatment products removal 824 (FIG. 8) and the fluid delivery channel 528 is connected to a source of fluid 820 (FIG. 8) with the help of the same connector 116 or by a separate connector. Operation of the facility for adipose tissue laser treatment products removal and the source of fluid synchronize with the operation of laser source and RF energy delivery.

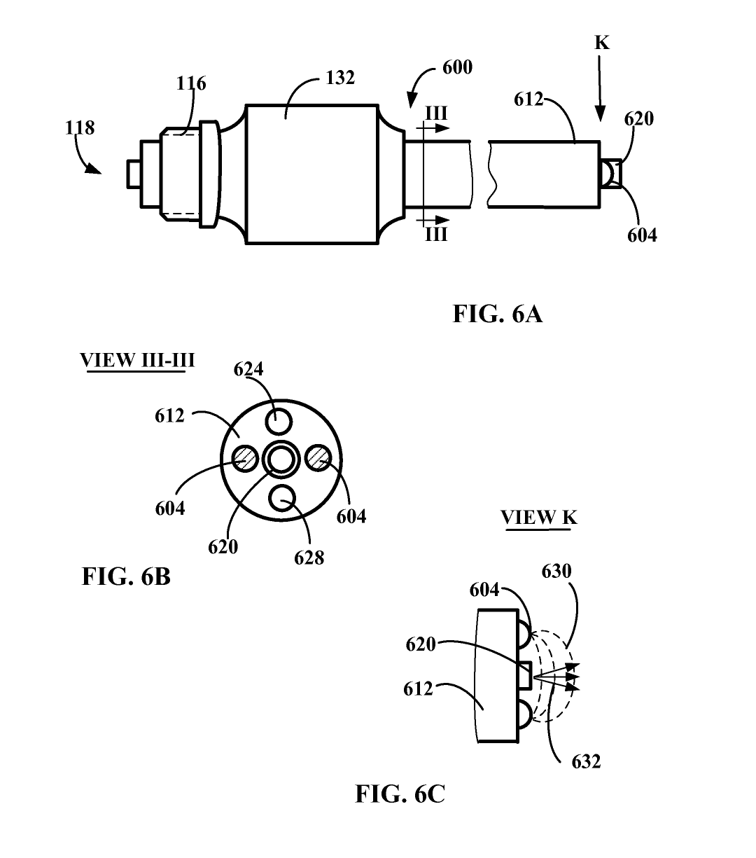

FIGS. 6A and 6B are schematic illustrations of a fifth exemplary embodiment of an energy-conveying needle with RF energy supplying electrodes. Needle 600 contains two, rod type electrodes 604, a light conducting guide 620, a fluid delivery channel 624 and adipose tissue treatment products removal channel 628, all incorporated into a common catheter-like structure 612. Light conducting guide 620 is connected to a source of laser radiation of suitable wavelength and power. If necessary, fluid may be supplied to the target volume (not shown) through delivery channel 624. Adipose tissue treatment products such as liquefied fat, if necessary, may be removed through removal channel 628. FIG. 6C illustrates operation of probe 600. Numeral 630 illustrates RF current lines and numeral 632, laser radiation irradiating the target tissue volume.

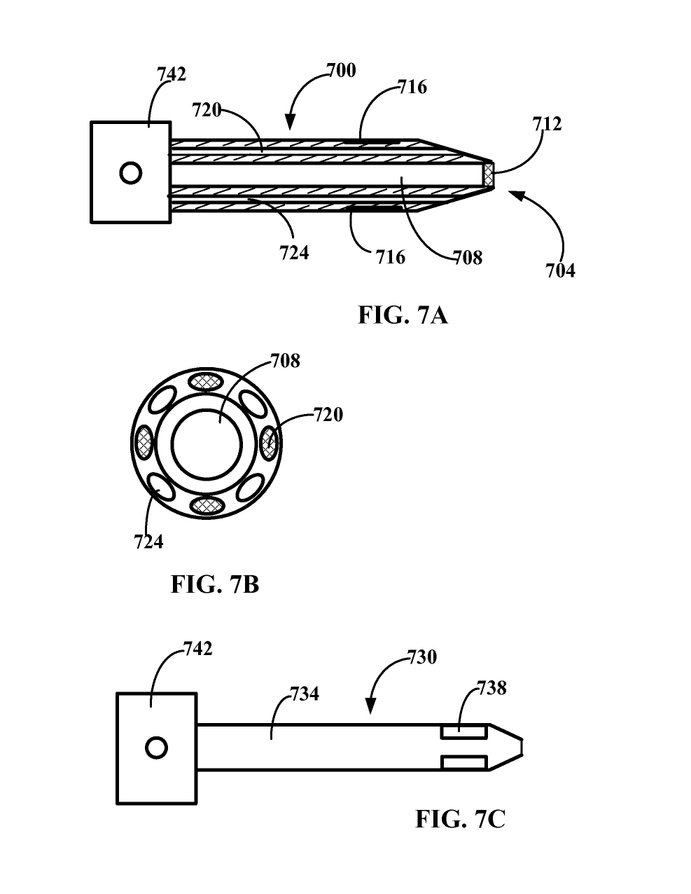

FIGS. 7A-7C, collectively referred to as FIG. 7, are schematic illustrations of a sixth exemplary embodiment of a flexible or rigid, hollow or solid energy-conveying needle 700. The emitting end 704 of light guide 708, which is introduced into the adipose tissue for treatment, is covered by a sapphire, diamond, or YAG window 712. During the course of liquefying adipose tissue, certain materials (termed carbonized materials) resulting from tissue with RF energy and high laser power interaction, deposit on end 708 of needle 700. These carbonized deposits increase laser light absorption by end 708 of needle 700 reducing the amount of laser radiation delivered to the target tissue volume. This deposit should be removed periodically. Increased laser power absorption in the carbonized deposit can increase local temperature at the first end 712 of needle 700 resulting in the needle damage. Sapphire, YAG, and diamond or similar materials are generally resistant to high temperature. Their use as a termination of the first end of the needle significantly improves the carbonization resistance and useful life of the needle.

Similar to the earlier disclosed exemplary embodiments, needle 700 includes one or more electrodes 716 deposited or built-in into the external surface of the needle. As shown in FIG. 7B, needle 700 may have channels 720 for fluid supply and channels 724 for liquefied fat and other adipose tissue laser treatment products removal and aspiration. In some embodiments, their may be one fluid conducting channel only and it may be used either for fluid delivery or adipose tissue treatment products removal.

FIG. 7C is an illustration of a needle 730, the body 734 of which is made completely of sapphire. Such a needle is more resistant than glass needles to deposition of carbonized laser treatment products. Electrodes 738 conforming to the shape of needle 730 may be incorporated in needle 730. A protective and insulating layer may cover the electrodes if necessary. Needles 700 and 730 may connect by their second end 742 with the help of an additional cable to a controller 818 (FIG. 8) or similar.

FIG. 8 is a schematic illustration of an apparatus for laser and RF assisted liposuction employing the present needle. Connector 116 connects needle 100 or 300 or any other needle described above via a cable 806 to a source of laser radiation 810 and a source of RF energy 814, which may be incorporated into a controller 818, or possibly stand-alone units. In addition, cable 806 may include at least one fluid conducting channel connecting the needle to a source of fluid 820 and/or adipose tissue treatment products removal facility 824.

In some embodiments, the needle is long enough to connect directly to a source of laser radiation and a source of RF energy 814. In such case, a separate cable 806 may include the RF conducting leads, which connect electrodes directly to the controller. Cooling fluid conducting and removal channels may be included in either of the cables. Controller 818 may operate the source of laser radiation 810 and the source of RF energy in a pulse or continuous radiation mode.

Controller 818 may further include a display 830 with a touch screen, or a set of buttons providing a user interface and synchronizing operation of the source of laser radiation 810 and the RF generator 814 with the operation of facility for adipose tissue treatment products removal facility 824 and a source of fluid 820.

When RF energy of proper value is applied to the adipose tissue, it heats the tissue and may liquefy it. Laser radiation of proper power and wavelength when applied to the adipose tissue may destroy fibrotic pockets releasing liquefied fat. The liquefied adipose tissue may be removed or may be left in the body, where it gradually dissipates. Application of each of the energies alone requires a significant amount of energy, which is associated with high cost. Generally, the energy provided by laser radiation is more costly than that of RF energy.

The present apparatus enables a method for adipose tissue laser treatment combining the RF energy and laser radiation. For treatment, needle 100 or any other needle described above is introduced into a target tissue volume 836 of adipose tissue 840. RF generator becomes operative to supply lower cost RF energy to the target volume and heat it to a desired temperature. A relatively small addition of laser energy or radiation is required to liquefy target volume of adipose tissue 836, destroy fibrotic pockets and release the liquefied fat. Both the RF energy and laser radiation may be delivered into the target tissue volume in a pulse or continuous mode and either simultaneously or subsequently in at least partially overlapping periods. RF energy delivered to the target tissue volume 836 heats the volume and laser radiation source 810 delivers additional tissue-destroying energy to target volume 836. Both laser and RF energies may cause controllable dermal collagen heating and stimulation.

Concurrently with the operation of the source of RF energy 814 and laser radiation source 810, the facility for adipose tissue treatment products removal 824 and, if necessary, fluid supply facility 820 become operative. The caregiver or apparatus operator moves the needle inserted in the tissue back and forth and periodically changes its angle of movement.

It is known that a number of wavelengths may be conducted through the same light guide. In order to facilitate the process of treatment location observation of tissue, an additional second laser, visible through skin/tissue laser, such as a HeNe laser may be coupled to needle 100 or cable 806. The HeNe laser, which is visible through skin, may assist the caregiver/operator in repositioning first end 108 of needle 100. Upon completion of treatment, needle 100 may be discarded. In an alternative embodiment, a temperature sensitive cream or temperature sensitive liquid crystal paste or film may be applied to the skin over the treated adipose tissue section. The paste/spread may be such as Chromazone ink commercially available from Liquid Crystal Resources/Hallcrest, Inc. Glenview Ill. 60026 U.S.A.

In yet another embodiment, laser beams from two laser sources with different wavelength could be used to optimize simultaneous fat destruction and blood haemostatis. The laser wavelengths may, for example, be 1.06 micrometer wavelength provided by NdYAG laser and a 0.9 micrometer wavelength provided by a laser diode. Another suitable set of wavelength is 1.064 micron and 0.532 micron. Such combination of laser wavelength reduces the bleeding, makes the fat removal procedure safer, and shortens the patient recovery time.

In still a further embodiment, following tissue heating or almost simultaneously with tissue heating by RF energy, a pulsed IR laser, for example a Ho—Tm (Holmium-Thulium) or Er:Yag laser generating pulses in sub-millisecond or millisecond range, may be applied to the same target tissue volume 836. During the laser pulse, the target tissue (cells and intercellular fluid) near the end 108 (FIG. 1) of needle 100 (or any other needle end) changes to overheated (high-pressure) gas forming expanding micro bubbles collapsing at the end of the pulse. Mechanical stress developed by that action may increase the rate of membrane of adipose cell disruption and release of liquefied fat from the cell. This opto-mechanical action of laser radiation combined with volumetric RF heating efficiently liquefies fat and makes fat removal/suction more efficient. The laser radiation pulse induces mechanical stress on cells in the target volume and delivers additional energy to the target volume that is sufficient for adipose tissue destruction.

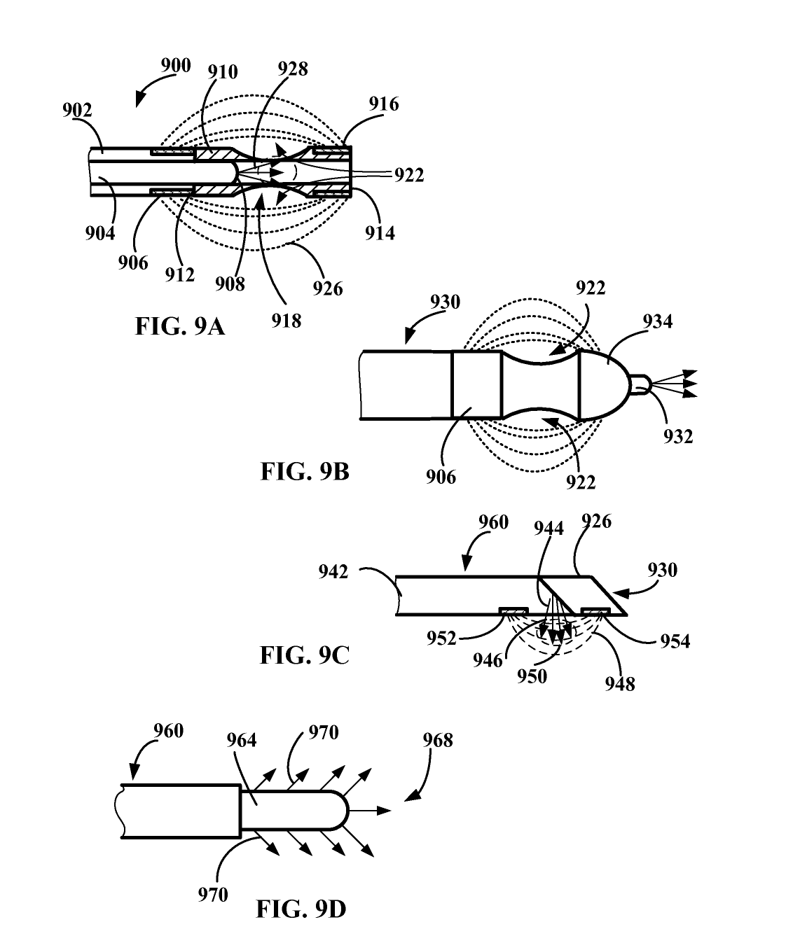

FIGS. 9A-9D are schematic illustrations of additional exemplary embodiments of the needle for laser and RF assisted liposuction. FIG. 9A illustrates a needle 900 having a jacket 902 and a light conducting body 904 made from electrically non-conductive material. A cylindrical electrode 906 is drawn over the radiation or energy-emitting end 908, of light conducting body 904. A cylindrical bushing 910 having a proximal end 912 and a distal end 914 is tightly fit over the light conducting body 904 or over jacket 902. Distal end 914 of bushing 910 is formed to receive a second electrode 916. Both electrodes, which may be concentric and coaxial electrodes, are connected to the source of RF energy 814 (FIG. 8). Bushing 910 features one or more openings 918 arranged on opposite sides of bushing 910. As needle 900 moves back and forth, it picks-up new portions of RF heated fat tissue, the flow of which is shown by lines 922. Lines 926 illustrate RF induced current and lines 928 illustrate schematically the laser radiation melting the fat. Laser radiation 928 is emitted into the fat volume located between electrodes 906 and 916 in a pulse or continuous radiation mode and provides additional energy for faster fat liquefaction. Needle 900 may include fluid conducting channels (not shown) for delivery or removal of fluids such as a cooling fluid, heating fluid, conductivity changing fluid, or products of adipose tissue treatment.

FIG. 9B illustrates a needle 930 including a protruding light guide 932 and electrode 934 having a shape that is easier to advance in a path formed in the adipose tissue by laser energy emitted through the end of light guide 932. Needle 930 may include fluid conducting channels (not shown) for delivery or removal of fluids such as a cooling fluid, heating fluid, conductivity changing fluid, or products of adipose tissue treatment.

FIG. 9C illustrates a needle 940 comprising a light guide 942 made from electrically non-conductive material or a layer of isolation placed over light guide 942. The first end 944 of needle 940 is formed to enable laser radiation 946 emissions in the direction of opening 946. Lines 948 indicate RF induced current heating a target volume 950 of the tissue. Laser radiation 946 is emitted into the same heated by RF volume 950 in a pulse or continuous radiation mode and provides additional energy for faster fat liquefaction. Electrodes 952 and 954 may be coated by a dielectric or be in direct contact with the tissue. An extender 956 may be attached to needle 940 for mounting electrode 954 on it. Alternatively, electrode 954 may be attached directly to needle 940.

FIG. 9D illustrates a needle 960 including a light conducting body 964, the first end 968 of which is shaped to generate a certain radiation distribution pattern illustrated by arrows 970 or diffuse laser power uniformly at the target treatment volume. The radiation-diffusing end would typically be 3 mm to 30 mm and such needle may be used, for example, at high laser power to avoid local overheating and needle tip carbonization. Needle 960 may be used for haemostasis.

FIG. 10 is a schematic illustration of the seventh exemplary embodiment of a laser radiation-conveying needle, which may be a disposable or reusable needle. Handle 132 (FIG. 1) is integral with an interim light guide, which is incorporated into cable 1004, and needle 1008 is implemented as a reusable/exchangeable or disposable part. Cable 1004 may include fluid supply channels and treated tissue debris removal channel. Relevant conductors supplying RF energy to electrodes 10012 could be incorporated in cable 1004. The disposable part 1008 may be connected to handle 132 by any known and suitable quick connection/removal connectors. Any one of the similar needle structures described herein could be used instead of disposable needle 1008.

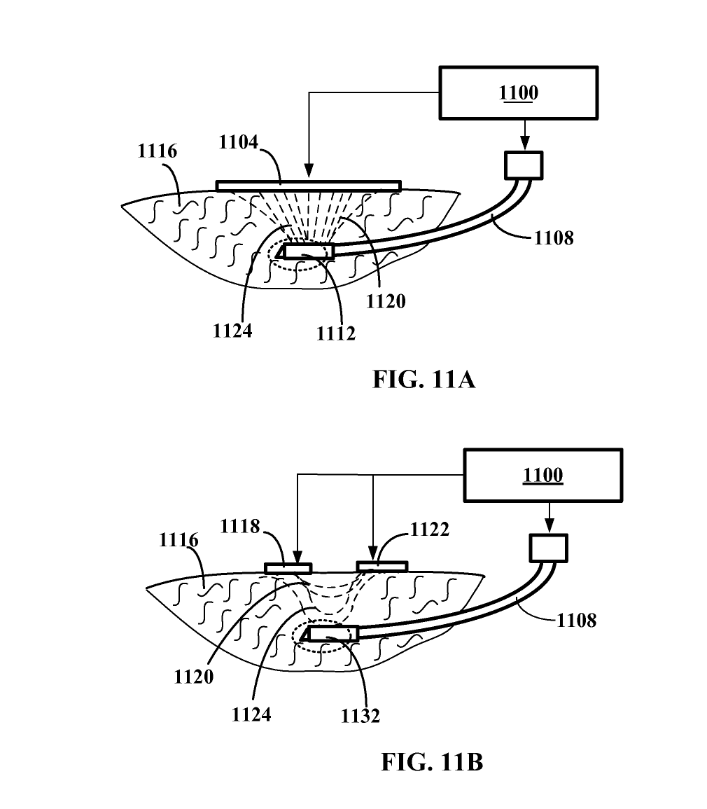

FIG. 11A is a schematic illustration of another exemplary embodiment of an apparatus for laser and RF assisted liposuction employing the present needle. The apparatus includes a controller 1100 similar to controller 818. It provides RF energy to bi-polar electrodes configuration that includes an external or first electrode 1104 and a needle 1108 similar to needles having a second electrode 1112 implemented as a cylinder integral with needle 1108. Both electrode 1104 and electrode 1112 may be coated by a dielectric providing capacitive coupling with tissue 1116 or have bare metal surface for conductive coupling with tissue 1116.

Needle 1108 is introduced subcutaneous into tissue 1116. Controller 1100 initiates supply of RF energy to electrodes 1104 and 1112. Electric current induced by the RF energy and shown by lines 1120 heats target tissue volume 1124 and laser radiation supplied through needle 1108 destroys the adipose tissue in target volume 1124. In this configuration, the density of RF energy is higher on internal electrode 1112. Electric current passes though all parts of tissue 1116, improves tissue texture and tightens tissue 1116. The configuration helps to break down and destroy adipose tissue and also shrink and contract it. The laser radiation may be provided by an NdYAG laser. The power of the radiation may be 0.5 watt to 50 watt.

FIG. 11B is a schematic illustration of an additional exemplary embodiment of an apparatus for laser and RF assisted liposuction employing the present needle. Two separate electrodes 1118 and 1122 replace the external electrode 1104. Needle 1108 may be terminated by electric current conducting termination 1132. In this embodiment, the electric current lines will close through the termination 1132.

The apparatus disclosed above may also be used for skin tightening. The needle is inserted subcutaneously into a patient so that the first end of the fiber is introduced within the tissue underlying the dermis. RF energy and laser source emit radiation of suitable power that are conveyed by the needle and the electrodes to the dermis, where the radiation causes collagen destruction and shrinkage within the treatment area.

The disposable needle described enables continuous adipose tissue treatment process, significantly reduces the treatment time, makes the subject treatment more comfortable and simplifies the treatment process.

While the exemplary embodiment of the needle, apparatus and the method of treatment has been illustrated and described, it will be appreciated that various changes can be made therein without affecting the spirit and scope of the needle, apparatus or method of treatment. The scope of the needle, apparatus and the method of treatment therefore, are defined by reference to the following claims:

1. A needle for adipose tissue treatment, said needle comprising:
one or more light conducting guides having a first end and a second end, said first end being operatively configured for introduction into adipose tissue and said second end adapted to connect to a source of laser energy; and
one or more electrodes adjacent to said first end and incorporated with said light conductive guide into a common structure.

2. The needle according to claim 1, wherein the electrodes are at least one selected from a group consisting of a ring type electrode, rod type electrode, or electrodes partially conforming to the needle shape.

3. The needle according to claim 1, wherein the surface of the electrodes is coated by a coating consisting of at least one selected from a group of coatings comprising a conductive surface coating or a dielectric surface coating.

4. The needle according to claim 1, further comprising at least one fluid conducting channel incorporated into the common structure.

5. The needle according to claim 4, wherein the fluid conducting channel conducts at least one fluid selected from a group of fluids consisting of a cooling fluid, heating fluid, conductivity changing fluid, or products of adipose tissue treatment.

6. The needle according to claim 1, wherein said second end connects to a source of laser energy directly or via a fiber optics cable.

7. The needle according to claim 6, wherein said laser energy conducting guide is a solid or a hollow guide.

8. The needle according to claim 1, wherein said laser energy conducting guide is a solid or a hollow guide.

9. The needle according to claim 1, wherein the electrodes are connected to source of RF energy.

10. The needle according to claim 1, further comprising a connection to a controller regulating the energy of the RF and laser source and removing or delivering fluids to the adipose tissue.

11. The needle according to claim 1, wherein at least one sensor is located at the first end of said needle or light conducting guide and wherein said sensor is a thermal sensor.

12. The needle according to claim 1, wherein the needle is a disposable or reusable needle.

13. An apparatus for adipose tissue treatment, said apparatus comprising:
a needle including at least one light guide and at least one RF electrode;
one or more sources of laser energy communicating in course of the operation with said needle; and
a source of RF energy operatively configured to provide RF energy to the electrodes.

14. The apparatus according to claim 13, wherein said source of laser energy operates in one of a pulse or continuous energy-emitting mode.

15. The apparatus according to claim 13, wherein said source of RF energy provides the RF energy to said electrodes in one of a pulse or continuous energy delivering mode.

16. The apparatus according to claim 13, wherein the needle further comprises at least one fluid-conducting channel.

17. The apparatus according to claim 13, further comprising a controller providing a user interface and synchronizing operation of said source of laser radiation, RF generator, and fluid delivery and treatment products removal through the fluid conducting channel.

18. The apparatus according to claim 17, wherein said controller further comprises at least one of a current or temperature feedback loops.

19. The apparatus according to claim 13, further comprising a temperature sensor located on said needle.

20. A method for adipose tissue treatment, said method comprising:
introducing a needle into a target volume of adipose tissue, said needle including:
a light guide, at least one RF electrode, and at least one fluid conducting channel;
delivering RF energy to the target volume to heat said volume; and
operating one or more laser sources to deliver tissue destroying energy to said target volume.

21. The method according to claim 20, wherein the RF energy and the laser energy are supplied to the target volume in at least partially overlapping periods.

22. The method according to claim 20, wherein at least one laser operates in a continuous operation mode and at least one laser operates in a pulse operation mode.

23. The method according to claim 20, further comprising providing means for visual observation of the tip of said needle in the adipose tissue location.

24. The method according to claim 20, further comprising delivering or removing through the fluid conducting channel at least one fluid selected from a group of fluids consisting of a cooling fluid, heating fluid, conductivity changing fluid, or products of adipose tissue treatment.

25. A method of tissue treatment, said method comprising:
applying a first electrode to the outer surface of skin and introducing subcutaneously a needle with a second electrode;
providing a radio frequency energy between said electrodes;
irradiating by laser radiation at least a volume of the tissue surrounding said second electrode, said radiation being conducted through said needle; and
changing the tissue state.

26. The method according to claim 25, wherein the change of the tissue state includes at least one of the effects from the group of effecting including adipose tissue destruction, shrinking, breakdown, and skin tightening.

27. The method according to claim 25, wherein the RF frequency is 100 Khz to 100 Mhz.

28. The method according to claim 25, wherein said laser radiation is applied concurrently or at least partially overlapping periods with the radio frequency.

29. The method according to claim 25, further comprising delivering or removing to the treated volume at least one of a group of fluids consisting of a cooling fluid, heating fluid, conductivity changing fluid, or products of adipose tissue treatment.

30. A method of adipose tissue treatment, said method comprising:
applying at least two electrodes to the patient skin;
generating a radio frequency field between said electrodes;
introducing subcutaneously a light guide and locating said guide such that at least a section of it is located in said radio frequency field; and
irradiating by laser radiation the part of said tissue located in said radio frequency field.

31. The method according to claim 30, wherein a combined action of said radio frequency and said laser radiation is changing said tissue state.

32. The method according to claim 30, wherein said treating the adipose tissue includes at least one of a group consisting of adipose tissue destruction, shrinking, breakdown, and skin tightening.

33. A method of lipo-sculpturing a segment of subject body, said method comprising:
providing at least two sources of electromagnetic energy located in distant regions of the electromagnetic energy spectrum;
delivering the energy generated by the first source by contact with the skin to a target volume of the tissue;
introducing subcutaneously said second electromagnetic energy source and locating it such that it delivers the energy generated by the second source to said target volume of the tissue;
coupling to said target volume energy emitted by both sources; and
changing the state of said target volume of the tissue.

34. A method of lipo-sculpturing a segment of human body according to claim 33, wherein said method includes contraction of at least collagen containing tissue.

35. A method of adipose tissue treatment, said method comprising:
applying electromagnetic radiation generated by two different electromagnetic radiation sources to a target volume of the tissue, where the first source of electromagnetic radiation is applied externally such that said radiation penetrates the skin surface and is concentrated in the target volume and the second source of electromagnetic radiation is applied to the same target volume by the second source located in said volume;
setting the energy level of the first source to a level insufficient to produce the desired treatment effect; and
setting the energy level of the second source to a level that when combined with the first source it is sufficient to produce the desired treatment effect.

36. The method according to claim 35, wherein said first source of energy is a source of radio frequency radiation.

37. The method according to claim 35, wherein said second source of energy is a source of infrared radiation.

38. The method according to claim 35, further comprising delivering or removing to the treated volume at least one of a group of fluids consisting of a cooling fluid, heating fluid, conductivity changing fluid, or products of adipose tissue treatment.

39. A method for adipose tissue treatment, said method comprising:
introducing a needle into a target volume of adipose tissue, said needle including;
a light guide operatively configured to deliver laser radiation to the target volume, at least one RF electrode operatively configured to deliver RF radiation to the target volume, and at least one fluid conducting channel;
delivering at least one of the radiations to the target volume to destroy the adipose tissue of the volume; and
operating a mechanism to remove from the treated volume radiation adipose tissue interaction products.
